(19) United States
(12) Patent Application Publication
(10) Pub. No.: US 2011/0172604 A1
(43) Pub. Date: Jul. 14, 2011

(54) HYBRID GUIDEWIRE

(75) Inventor: Justin Wolfe, Lawrenceville, GA (US)

(73) Assignee: C. R. BARD, INC., Murray Hill, NJ (US)

(21) Appl. No.: 13/063,172

(22) PCT Filed: Sep. 11, 2009
(86) PCT No.: PCT/US2009/056639

§ 371 Date: Mar. 9, 2011

Related U.S. Application Data
(60) Provisional application No. 61/096,741, filed on Sep. 12, 2008.

(51) Int. Cl. A61M 25/09; B21C 23/00; B21B 45/00

(52) U.S. Cl. 604/164.13; 72/253.1; 72/46

(57) ABSTRACT

A hybrid guidewire, a method for manufacturing the hybrid guidewire including co-extruding a core inside a sheath in bulk, cutting the core and sheath to a desired length, and shaping a distal and proximal portion of the guidewire depending on the desired application.

### PRIORITY

This application claims the benefit of priority to U.S. Provisional Patent Application No. 61/096,741, filed Sep. 12, 2008, which is incorporated by reference into this application as if fully set forth herein.

### FIELD

The present invention relates generally to guidewires, including guidewires suitable for catheter-based medical procedures, and to manufacturing methods for hybrid guidewires.

### BACKGROUND

A guidewire is often used to guide a catheter along a body lumen. The guidewire may extend many feet into the body lumen and may be used to predetermine the path of the catheter. Because the guidewire is in sliding contact with tissue and other instruments, much of the length of the guidewire requires lubricity and durability. A guidewire may be composed of a durable metal core coated or jacketed with a lubricious sheath. Currently, hybrid guidewires are manufactured at high cost. The guidewires are jacketed individually, where the process of placing the jacket on the guidewire core is the most cumbersome and expensive process. Current processes comprises either shrink wrapping a jacket, extruding discrete lengths, or wire windings the wire core.

Guidewire devices and manufacturing methods are described, for example, in U.S. Pat. No. 5,452,726 (titled “Intravascular Guidewire and Methods for Manufacture Thereof,” issued Sep. 26, 1995), U.S. Pat. No. 6,251,086 (titled “Guidewire With Hydrophilically Coated Tip,” issued Jun. 26, 2001), U.S. Pat. No. 5,924,998 (titled “Guidewire With Hydrophilically Coated Tip,” issued Jul. 20, 1999), U.S. Pat. No. 6,656,134 (titled “Guidewire With Hydrophilically Coated Tip,” issued Dec. 2, 2003), U.S. Pat. No. 7,001,345 (titled “Guidewire,” issued Feb. 21, 2006), U.S. Pat. No. 5,281,203 (titled “Guidewire and Sheath for Single Operator Exchange,” issued Jan. 25, 1994), and U.S. Pat. No. 6,612,998 (titled “Guidewire with Marker Sleeve,” issued Sep. 2, 2003), each of which is incorporated by reference into this application as if fully set forth herein.

### SUMMARY

A method for manufacturing a hybrid guidewire is disclosed, including co-extruding a core inside a sheath in bulk, cutting the core and sheath to a desired length, and shaping a distal portion of the guidewire depending on the desired application. The proximal portion may also be shaped. The proximal and distal portions may be shaped by reducing the very distal end and very proximal end down to a constant diameter. The shaping may continue with tapering a proximal tapered portion and a distal tapered portion between the end portions and a central portion of the guidewire. The shaping may be accomplished by grinding the co-extruded core and sheath. The proximal portion may be covered by a pre-stressed heat shrinking tube, while the distal portion may be covered by a coil attached to a distal shoulder created by removing part of the sheath between the distal tapered section and the central section of the guidewire. The coil may be coated in a hydrophilic polymer.

### BRIEF DESCRIPTION OF THE DRAWINGS

FIG. 1A illustrates a profile of an exemplary hybrid guidewire according to embodiments of the invention.

FIG. 1B illustrates a cut-away view along the longitudinal axis of the exemplary hybrid guidewire of FIG. 1A.

FIG. 2A represents a flow diagram of the manufacturing process according to embodiments of the invention.

FIG. 2B represents a flow diagram of additional manufacturing processes according to alternate embodiments of the invention.

FIG. 3A illustrates representative guidewires at each manufacturing process represented in FIG. 2A, according to embodiments of the invention.

FIG. 3B illustrates representative guidewires at each manufacturing process represented in FIG. 2B, according to alternate embodiments of the invention.

### DETAILED DESCRIPTION

The following description should be read with reference to the drawings, in which like elements in different drawings are identically numbered. The drawings, which are not necessarily to scale, depict selected embodiments and are not intended to limit the scope of the invention. The description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. The term “substantial” indicates at least a portion of, which may include the entirety. The term “body” may indicate any suitable host, such as, for example, a human body including internal body cavities or an animal including mammalian bodies.

Embodiments of the present invention co-extrude a core in bulk with a single sheath. This allows for simpler processing than co-extruding discrete lengths or individually fitting shrink wrap on individually cut cores. The proximal end of the guidewire may be ground for easier insertion into a scope. The distal end may be ground to produce a flexible atraumatic tip. Shrink tube may also be added over discrete parts of the guidewire, such as the proximal end to provide the desired degree of lubricity. The preferred length and placement of shrink tube allows the guidewire to be manufactured for significantly less cost. A coil over the distal end may be added to enhance distal flexibility of the guidewire, which may also be coated with a hydrophilic polymer. The proximal shaft section may include grooves to enhance dry lubricity.

Although embodiments of the invention are typically described in terms of hybrid guidewires, the invention is not so limited. Aspects of the invention may be applied to intraluminal guidewires, for example intravascular guidewires. The guidewire produced by embodiments of the described manufacturing process may be used, for example, in various surgical procedures to guide a medical device through conduits in the body. The guidewires produced from the described embodiments may be used alone or in conjunction with other devices, such as a catheter.

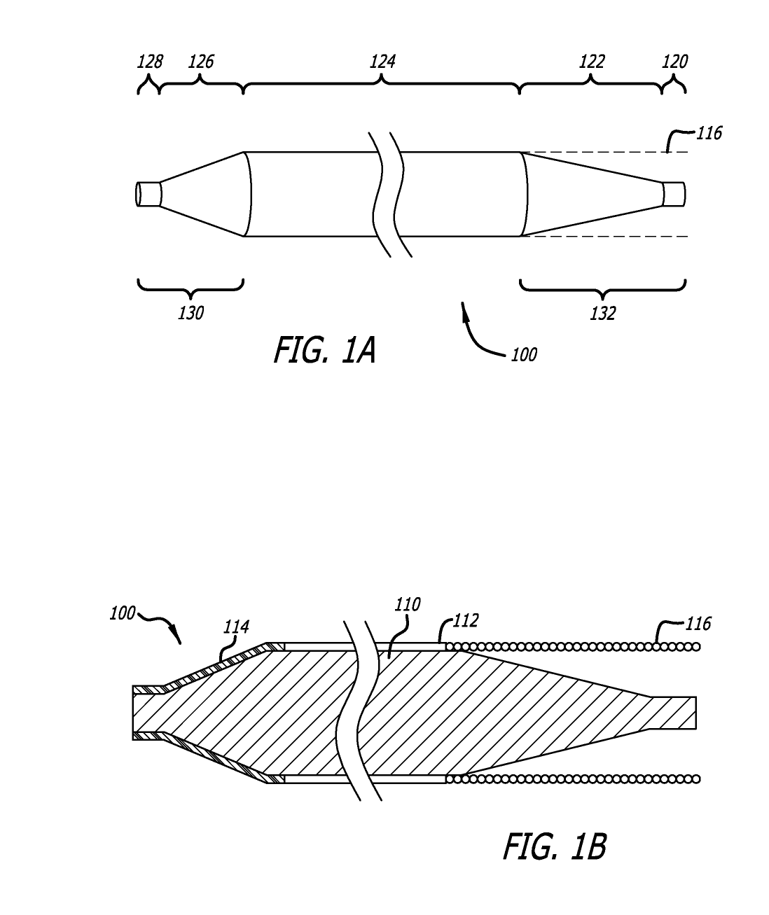

FIG. 1 illustrates an exemplary hybrid guidewire 100. FIG. 1A illustrates a profile view of a representative guidewire 100, while FIG. 1B illustrates a cutaway view along the longitudinal axis, both of which include features according to embodiments of the invention. The core 110 may be a solid wire made of a strong, flexible material, such as metal, e.g. Nitinol. The length and diameter of the core 110 may change depending on the application or procedure to be performed. The core 110 may have a varied external diameter and, according to certain embodiments, may be tapered at locations along the guidewire 100.

For example, the distal end portion 120 of the core 110 may have a constant diameter, followed proximately by a tapered distal portion 122, which may gradually increase the diameter of the core 110. The tapered distal portion 122 may or may not be a uniform transition from the smaller diameter of the distal end portion 120 to the larger diameter of the central portion 124. A tapered proximal portion 126 of the guidewire 100 may then taper after a substantially constant diameter of the central portion 124, reducing the diameter from the central portion 124 to the diameter at the proximal end portion 128. The final diameter of the guidewire 100 at the proximal end portion 128 does not have to be the same as the diameter at the distal end portion 120. According to various embodiments, the diameter of the distal end portion 120 is less than the diameter of the proximal end portion 128 to provide a greater flexibility at the distal end. The tapered portions 122 and 126 provide increasing levels of flexibility as the core diameter is reduced. The flexibility enhances maneuverability of the distal end through tortuous body lumens, while assisting in loading the guidewire at the proximal end.

All or part of the core 110 may be surrounded by a material having a surface with a reduced coefficient of friction compared to the core 110 surface, such as a fluoropolymer. The lower friction material, such as plastic, may reduce the friction of the majority of the guidewire 100 to permit easier insertion and manipulation of the guidewire within the body. The plastic may be applied by co-extrusion over the core material, heat shrinking pre-stressed tubing materials, or a combination of both over different sections of the core. For example, in one embodiment, the core 110 may include a sheath 112 substantially surrounding the longitudinal length of the central portion 124. The sheath 112 may be a plastic tube, such as a fluoropolymer, e.g. polytetrafluoroethylene (PTFE), polyvinylidene fluoride (PVDF), or fluorinated ethylene propylene (FEP). The sheath 112 may be co-extruded over all or a portion of the core 110. In addition, the proximal region 130 may be separately covered by a jacket 114, which may be plastic that may be or may not be the same material as the sheath 112. For example, the jacket 114 may be a pre-stressed plastic tube of fluoropolymer. The jacket 114 may be heat shrunk around a substantial section of the proximal region 130 and may cover a part of the central portion 124.

According to some embodiments, the sheath 112 may have a plastic exterior with a coefficient of friction that may be approximately half of the coefficient of friction of the exposed core. The lower coefficient of friction permits the guidewire to more easily pass through conduits and body lumens.

In one embodiment, a coil 116 may surround at least a portion of the core 110 at the distal region 132. Coil 116 is represented as a dotted line in FIG. 1A. Coil 116 may be a metal wire, helically wrapped at approximately constant diameter. For example, the coil 116 may substantially surround the distal region 132 of the core 110. The coil 116 may be coupled to the core 110 by welding, bonding, brazing, soldering, adhering, crimping, or by other known methods. The outer diameter of the coil 116 preferably matches the outer diameter of the core 110 at the central portion 124, or the sheath 112, if present, to create a smooth transition. The coil may be radiopaque for better viewing during a medical procedure. A portion or all of the finished guidewire may also be coated with a hydrophilic polymer. According to certain embodiments, a substantial length of the coil 116 is coated to make the surface highly lubricious when it comes in contact with a fluid, such as blood or urine. In addition, the distal and proximal ends of the wire may be polished or potted with UV epoxy for cosmetic purposes.

For example, in one embodiment, the core 110 is surrounded by a sheath 112 along the central portion 124, a coil 116 around the distal region 132, and a jacket 114 along the proximal region 130. The core 110 may be made of Nitinol, the sheath 112 and jacket 114 may be PVDF, and the coil 116 may be stainless steel. The core 110 may be approximately 50 inches to approximately 60 inches long, for example about 59 inches, with a diameter of approximately 0.005 inches to approximately 0.05 inches. The distal end portion 120, approximately the first one to two inches of the core 110, may have a generally constant diameter of about 0.006 inches. Then, the tapered distal portion 122 may transition between the about 0.006 inch diameter of the distal end portion 120 to about 0.026 inch diameter of the central portion 124 over approximately two to six inches, and preferably over approximately two to four inches. The about 0.026 inch diameter section may continue for approximately 40 to approximately 55 inches along the central portion 124, until the transition to the proximal taper begins. The tapered proximal portion 126 may be approximately two to six inches, and is preferably four to six inches, and may taper from about 0.026 inches down to about 0.010 inches at the proximal end portion 128 of the core 110. The proximal end portion 128 may extend proximal of the tapered proximal portion 126 at a generally contact diameter of about 0.010 inches for approximately one to two inches. A stainless steel coil may surround a substantial portion of the distal portion 132 and may be coupled to the core 110 via welds or UV epoxy. A sheath 112 of a fluoropolymer, such as PVDF, may surround a substantial portion of the central portion 124, while a jacket 114 of PVDF may surround a substantial portion of the proximal region 130. The transition of the guidewire outer diameter at the junction of the coil 116 with the sheath 112 and at the jacket 114 with the sheath 112 is approximately constant, about 0.026 inches, for a smooth transition between each region.

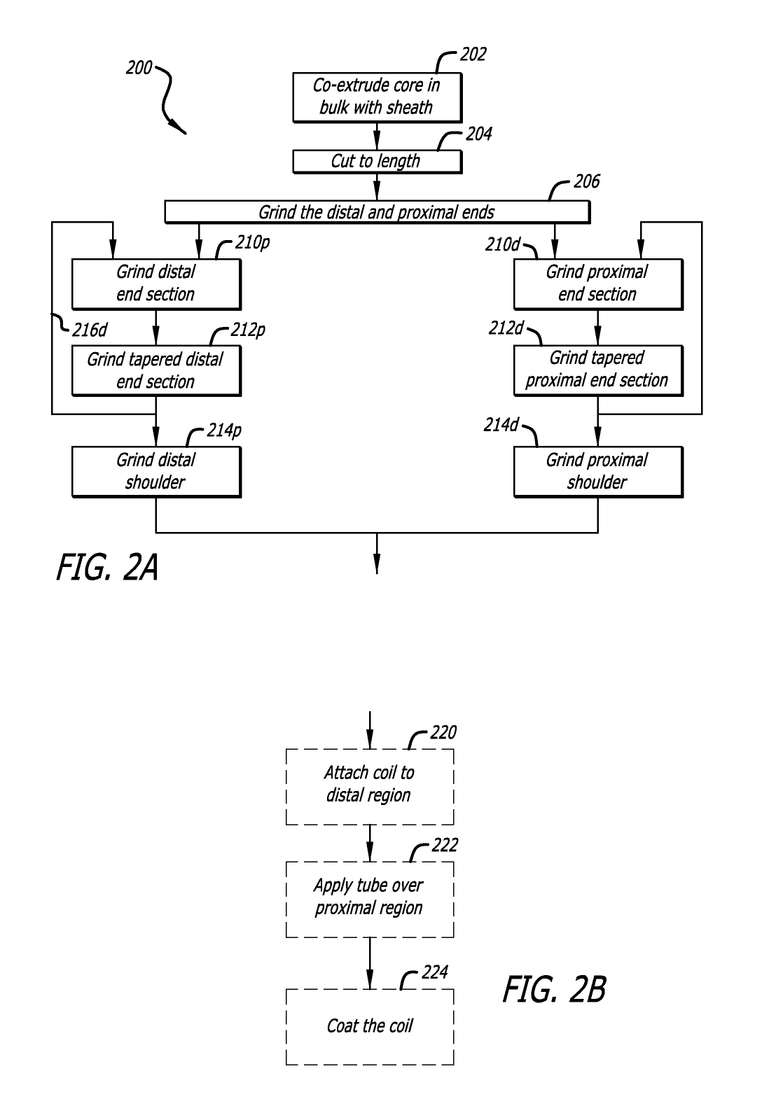

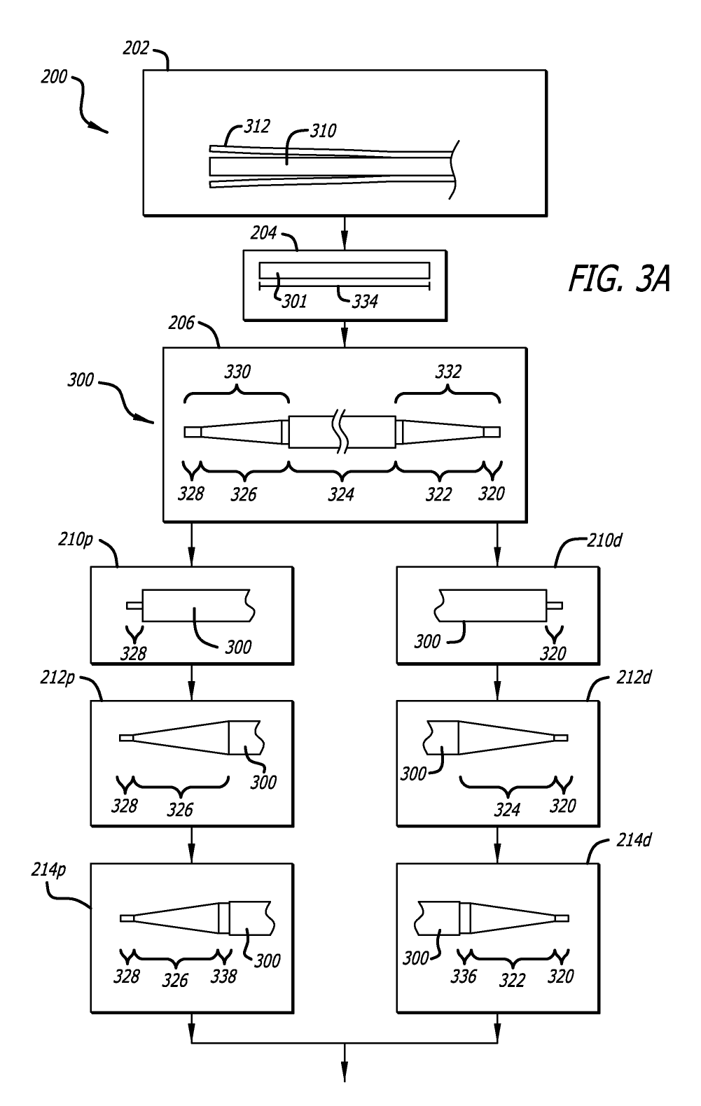

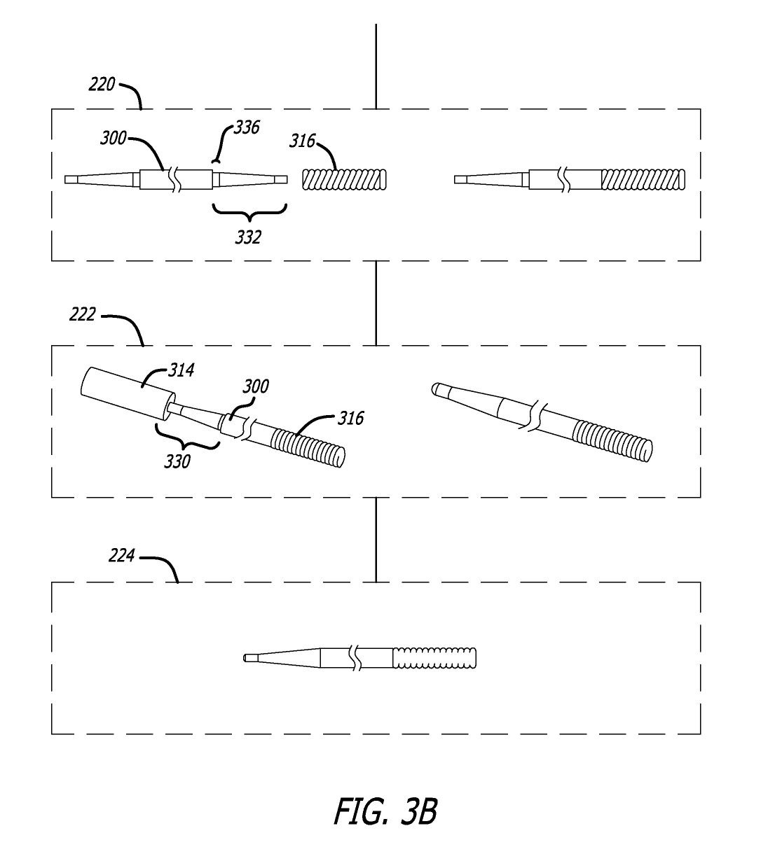

FIG. 2A illustrates a representative flow diagram of one embodiment of a manufacturing process 200 for the guidewire described herein. FIG. 2B illustrates representative optional manufacturing processing for the guidewire according to embodiments of the process. FIGS. 3A and 3B illustrate an exemplary guidewire 300 as it progresses through each part of the described methods, according to embodiments of the invention, as represented by FIGS. 2A and 2B. Though presented in a specific sequence, various parts may be carried out in different order, or combined or separated into more or less sub-procedures; some sub-procedures may be skipped completely (generally indicated in a dashed line, but not necessarily), while others may be performed simultaneously. For example, blocks 220, 222, and 224 can be reordered as needed. Grinding each end of the guidewire may also be performed at different times. The proposed method will simplify the current process, which may comprise individually placing and shrink wrapping a jacket on each core, and thereby reduce the complication and cost of the manufacturing process.

First, at block 202, the core 310 is co-extruded in bulk with a single plastic sheath 312. The co-extrusion of the core wire in bulk allows for simpler processing than co-extruding discrete lengths. The core 310 may preferably be Nitinol, while the sheath 312 may be plastic, preferably a fluoropolymer, such as PTFE, PVDF, or FEP. More particularly, the sheath 312 may preferably be PVDF, as it has the best combination of material properties and processing temperature. The sheath 312 may be extruded with axially oriented grooves to reduce the frictional properties of the shaft of the guidewire 300. The diameter of the core 310 may be approximately 0.018 to 0.030 inches, and approximately 0.025 to 0.038 inches with the sheath 312, following the extrusion.

Second, at block 204 the guidewire 300 may be cut to approximately the finished length 334. Generally, the finished length 334 may be approximately 50 to 60 inches. This leaves discrete guidewire blanks 301 for further processing into desired shapes and configurations for the individually desired application.

Third, at block 206, the distal region 332 and proximal region 330 may be ground through both the sheath 312 and the core 310, as needed. The distal region 332 and proximal region 330 may be be ground to the desired dimensions and shapes required by individual applications. The regions may be ground to generally uniform diameter over a section of the guidewire 300, such as at the distal end portion 320 or proximal end portion 328. The regions may alternatively or in conjunction be ground at a varying diameter over a length of the guidewire 300, such as for the tapered distal portion 322 or the tapered proximal portion 326. Multiple sections of constant and varying diameters may be ground in a step-wise fashion as required by the application.

For example, the distal end portion 320 may be ground, through both the sheath 312 and the core 310, with a constant diameter of about 0.006 inches over the last one to two inches, as indicated in block 210d. The next grinding, block 212d, may shape the tapered distal portion 322. The tapered distal portion 322, proximal the distal end portion 320, may be ground through the sheath 312 and core 310 with a taper upward from 0.006 inches to the diameter of the core 310 (about 0.018 inches to 0.030 inches) over a length of approximately two to six inches, depending on the desired stiffness. The last part of the distal grind, the distal shoulder 336, may be through the sheath 312 only, and therefore be generally constant diameter of about the core 310 diameter of about 0.018 inches to about 0.030 inches, and may leave a shoulder approximately 0.050 inches to approximately 0.25 inches long.

The proximal region 330 may also be shaped to create a profile to more easily insert into a scope. The proximal shaping may be done before, after, or simultaneous with the distal shaping. First, block 210p, the proximal end portion 328 may be ground with to a generally constant diameter of about 0.010 inches for the last one to two inches thereof. The next length, the tapered proximal portion 326, may taper upward from 0.010 inches to the diameter of the core 310 over a length of two to six inches, block 212p. The last part of the proximal grind, block 214p, may be through the sheath 312 only and leave a proximal shoulder 338 of about 0.050 inches to about 0.25 inches for shrink wrapping a cover over the proximal ground core.

The above methods, particularly blocks 210d and 212d, may be repeated as needed to manufacture additional generally constant diameter sections and tapered sections over specific lengths of the guidewire 300. Other methods, such as chemical washes, polishes, or combinations thereof, may alternatively be used to grinding.

In one embodiment, after grinding, a coil 316 may be attached over the distal region 332 of the guidewire 300, at block 220. The distal shoulder 336, as described previously, provides an attachment surface for the coil 316. The coil 316 may be adhered, welded, or attached through other methods known in the art.

In one embodiment, block 222, a tube 314 may be attached over the proximal region 330 of the guidewire 300. The tube 314 may be heat shrunk to tightly fit around the proximal region 330 and cover the ground surface of the guidewire 300 along the proximal region 330. The tube 314 may be of the same material as the sheath 312. By covering only the ground proximal section of the guidewire 300, only two to six inches of shrink tube may be required, allowing the guidewire 300 to be manufactured for significantly less cost. The tube 314 may overlap on the proximal shoulder 338 to ensure a smooth transition between the sheath 312 and the tube 314 by moving the junction along the constant diameter of the central section 324 and away from the junction created between the tapered proximal portion 326 and the central section 324. Additionally, block 224, the coil 316 may be coated with a hydrophilic material, such as a hydrophilic polymer, in order to provide enhanced lubriciousness. For example, silicone coating or other lubricious material(s) may be used, and may be applied via coating, dipping, or any other standard method.

A primer coat may be disposed between the jacket and the core. A primer coat may also be disposed between the sheath and the core. A primer coat may also be disposed between the coil and the core. The primer coat may be a polyurethane-based primer.

Embodiments described herein include manufacturing methods for hybrid guidewires. The described method may improve manufacturing processes to reduce the cost of a hybrid guidewire. Embodiments of the manufacturing methods may simplify the processing of guidewires, thereby manufacturing the hybrid guidewire for significantly less cost. While the design has been described in terms of particular variations and illustrative figures, those of skill in the art will recognize that the design is not limited to the variations or figures described. In addition, where methods and steps described above indicate certain events occurring in certain sequence, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

1. A method of manufacturing a hybrid guidewire, comprising:
co-extruding a core inside a sheath in bulk, the co-extruded core and sheath having a first length;
cutting the co-extruded core and the sheath to a desired second length less than the first length to provide a blank; and
shaping a distal portion of the blank.

2. The method according to claim 1, further comprising the step of shaping a proximal portion of the blank.

3. The method according to claim 2, wherein the step of shaping the proximal portion of the blank further comprises reducing a proximal end portion to a generally constant proximal end diameter, smaller than an original diameter of the blank.

4. The method according to claim 3, wherein the step of shaping the proximal portion of the blank further comprises tapering a tapered proximal portion to smoothly transition between the original diameter of the blank and the generally constant proximal end diameter.

5. The method according to claim 3, wherein the step of reducing the proximal end portion includes grinding the proximal end portion to the generally constant proximal end diameter.

6. The method according to claim 4, wherein the step of tapering the tapered proximal portion includes grinding.

7. The method according to claim 4, further comprising removing a portion of the sheath between the tapered proximal portion and an unshaped portion of the blank creating a proximal shoulder.

8. The method according to claim 2, further comprising covering the proximal portion.

9. The method according to claim 8, wherein the step of covering the proximal portion includes covering with a pre-stressed heat shrinking fluoropolymer tube.

10. The method according to claim 1, wherein the step of shaping the distal portion further comprises reducing a distal end portion to a generally constant distal end diameter, smaller than an original diameter of the blank.

11. The method according to claim 10, wherein the step of shaping the distal portion further comprises tapering a tapered distal portion to smoothly transition between the original diameter of the blank to the generally constant distal end diameter.

12. The method according to claim 10, wherein the step of reducing the distal end portion includes grinding the distal end portion to the generally constant distal end diameter.

13. The method according to claim 11, wherein the step of tapering the tapered distal portion includes grinding.

14. The method according to claim 11, further comprising removing a portion of the sheath between the tapered distal portion and an unshaped portion of the blank creating a distal shoulder.

15. The method according to claim 14, further comprising attaching a stainless steel coil coated with a hydrophilic polymer to the distal shoulder.

16-17. (canceled)

18. The method according to claim 1, wherein the core is a Nitinol wire and the material of the sheath is selected from the group consisting essentially of PTFE, PVDF, FEP, and combinations thereof.

19-21. (canceled)

22. The method according to claim 1, wherein the desired length is approximately 50 to 60 inches.

23-26. (canceled)

27. A guidewire comprising:
an elongated core having a taper at both its proximal and distal ends;
a jacket comprising a fluoropolymer disposed over the elongated core on at least a portion of the proximal end thereof;
a radiopaque coil surrounding the elongated core on at least a portion of the distal end thereof; and
a hydrophilic coating disposed over at least a portion of the radiopaque coil, wherein the outer diameter of the coil-wrapped elongated core is substantially equal to the outer diameter of the jacketed elongated core.

28. The guidewire according to claim 27, wherein a polyurethane-base primer coat is disposed between the jacket and the elongated core, and between the radiopaque coil and the elongated core.

29-31. (canceled)

32. The guidewire according to claim 27, wherein the elongated core comprises Nitinol, the radiopaque coil comprises stainless steel, and the fluoropolymer is polytetrafluoroethylene.

33-35. (canceled)
